(19) United States
(12) Patent Application Publication
(10) Pub. No.: US 2022/0062344 A1
(43) Pub. Date: Mar. 3, 2022

(54) COMPOSITIONS AND METHODS FOR PROVIDING HEALTH BENEFITS IN AN ANIMAL

(71) Applicant: Société des Produits Nestlé S.A., Vevey (CH)

(72) Inventors: Ping Yu, St. Louis, MO (US); Yuanlong Pan, Chesterfield, MO (US); Rondo Middleton, CREVE COEUR, MO (US); Janet JACKSON, COLUMBIA (MO)

(21) Appl. No.: 17/398,145

(22) Filed: Aug. 10, 2021

Related U.S. Application Data
(60) Provisional application No. 63/074,368, filed on Sep. 3, 2020.

(51) Int. Cl. A61K 35/20; A23C 9/20; A23K 50/48; A23K 10/20

(52) U.S. Cl. CPC A61K 35/20; A23K 10/20; A23K 50/48; A23C 9/206

(57) ABSTRACT

A method of enhancing vaccine efficacy, treating or preventing an allergy, or treating or preventing an autoimmune disease in an animal can comprise administering to the animal a composition comprising colostrum.

### CROSS REFERENCE TO RELATED APPLICATION

This application claims priority to U.S. Provisional Application Ser. No. 63/074,368 filed Sep. 3, 2020 the disclosure of which is incorporated in its entirety herein by this reference.

### BACKGROUND

Infection is a major health issues affecting human and companion animals, especially in senior people and companion animals. One approach to prevent or reduce infection is vaccination. Unfortunately, age-related changes of the immune system results in impaired vaccination response, which greatly reduces the efficacy of vaccination, leading to increased incidence and severity of infections in the elderly. Therefore, there is urgent need to develop nutritional solutions to boost vaccination response in senior people and animals including pets.

Additionally, allergy is a major immune disorder affecting people and pets, which adversely affects quality of life in people and pets. Currently, solutions for allergy usually rely on various drugs which can have undesirable side effects. Nutritionally, omega-3 poly-unsaturated fatty acids can have some anti-allergy benefits, but they are expensive. Therefore, there is need to develop alternative nutritional solutions to treat or prevent allergies in people and animals including pets.

Further, autoimmune diseases are also a major immune disorder affecting people and animals, which adversely affects quality of life in people and animals including pets. Currently, a common solution for autoimmune diseases is administration of various drugs which can have many side effects and can be expensive. Nutritionally, there is no effective strategy to treat or prevent autoimmune diseases in human and animals including pets.

There is, therefore, a need for new treatments that can overcome these challenges.

### SUMMARY

The present disclosure relates generally to methods and compositions for providing health benefits in an animal using colostrum.

In one embodiment, a method of treating or preventing an allergy, or treating or preventing an autoimmune disease, in an animal, can comprise administering to the animal a composition comprising colostrum.

In another embodiment, a method of enhancing vaccine efficacy in a senior animal can comprise comprising administering to the animal a composition comprising colostrum.

Additional features and advantages are described herein and will be apparent from the following Detailed Description.

### DETAILED DESCRIPTION

### Definitions

As used in this disclosure and the appended claims, the singular forms “a,” “an” and “the” include plural referents unless the context clearly dictates otherwise. Thus, for example, reference to “a composition” or “the composition” includes two or more compositions. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative, and are not exclusive or comprehensive.

The term “animal” means any animal that can benefit from the present compositions, including human, avian, bovine, canine, equine, feline, hircine, lupine, murine, ovine, or porcine animals. In one aspect, the animal can be a mammal. In another aspect, the animal can be a companion animal.

The term “companion animal” means domesticated animals such as cats, dogs, rabbits, guinea pigs, ferrets, hamsters, mice, gerbils, horses, cows, goats, sheep, donkeys, pigs, and the like. In one aspect, the companion animal can be a canine. In another aspect, the companion animal can be a feline.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein. Additionally, for example, if the average lifespan for a breed of cat is 15 years, then a cat within that breed greater than 7.5 years old would be considered “aging” for the purposes herein. In an embodiment, the compositions and methods disclosed herein involve a senior animal, e.g. a senior dog or a senior cat. Animals, such as dogs and cats, are considered senior in the last 25% of their lives. As noted herein, the life span of a dog or a cat depends on its size and/or its breed, and a senior dog or senior cat can be determined based on the above calculation but using the numerical value of 75% such that the age threshold is exceeding 75% of the average lifespan.

As used herein, “about” is understood to refer to numbers in a range of numerals, for example the range of −10% to +10% of the referenced number, within −5% to +5% of the referenced number, or in one aspect, within −1% to +1% of the referenced number, and in a specific aspect, within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the food composition unless expressed otherwise. When reference is made to the pH, values correspond to pH measured at 25° C. with standard equipment. An “amount” can be the total amount of the referenced component per serving of the composition or per distinct unit of the composition and/or can be the weight percentage of the referenced component by dry weight. Moreover, an “amount” includes zero; for example, the recitation of an amount of a compound does not necessarily mean that the compound is present, unless followed by a range that excludes zero.

The terms “pet food,” “pet food product” and “pet food composition” mean a product or composition that is intended for ingestion by a canine or feline that provides at least one nutrient to the animal. Further in this regard, these terms mean that the product or composition is in a form ready for consumption and is not merely an intermediate from which a consumable product or composition is made, although other food compositions can be added in some embodiments, such as a dietary supplement. The term “pet food” means any food composition intended to be consumed by a canine or feline.

The term “effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In one embodiment, the present colostrum can be present in an effective amount enhancing vaccine efficacy, treating or preventing allergies, and/or treating or preventing autoimmune diseases.

The dosages expressed herein are in milligrams per kilogram of body weight per day (mg/kg/day) unless expressed otherwise.

The term “long-term administration” means periods of repeated administration or consumption in excess of one month. Periods of longer than two, three, or four months can be used for certain embodiments. Also, more extended periods can be used that include longer than 5, 6, 7, 8, 9, or 10 months. Periods in excess of 11 months or 1 year can also be used. Longer term use extending over 1, 2, 3, or more years are included in the invention. For certain aging canines, the canine will continue consuming on a regular basis for the remainder of its life. This can also be referred to as consumption for “extended” periods.

The term “regular basis” or “regular administration” means at least monthly dosing with the compositions or consumption of the compositions, and in one aspect, means at least weekly dosing. More frequent dosing or consumption, such as twice or three times weekly, can be performed in certain embodiments. Still, in other embodiments, regimens can be used that comprise at least once daily consumption. A frequency, regardless of whether expressly exemplified herein, that allows maintenance of a desired activity level of the measured compound within acceptable ranges can be useful herein. The skilled artisan will appreciate that feeding amounts will be a function of the composition that is being consumed or administered as well as the animal consuming the food, and some food compositions may require more or less frequent administration to maintain a desired activity level of the measured compound.

The relative terms “improve,” “increase,” “enhance,” “decrease” and the like refer to the effects of the composition disclosed herein (a composition comprising colostrum) relative to a composition having a lower amount or lacking such compositional elements, but otherwise identical.

A “blended” composition merely has at least two components having at least one different characteristic relative to each other. In one aspect, moisture content and water activity can be different in the context of the present disclosure. In this regard, description of a composition as “blended” does not imply that the blended composition has been subjected to processing sometimes referenced as “blending,” namely mixing components so that they are indistinguishable from each other, and, in one aspect, such processing is avoided when mixing one component with the other components to form a blended composition (e.g., mixing a dry component with a wet or semi-moist component). Further in this regard, in a blended composition each of the at least two components having at least one different characteristic relative to each other can retain their distinct identity and appearance.

“Wet food” means a pet food having a moisture content from about 50% to about 90%, and in one aspect, from about 70% to about 90%. “Dry food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%.

“Kibbles” is used synonymously with “chunks” herein and both terms mean pieces of dry or semi-moist pet food which can have a pellet shape or any other shape and can be made by slicing a food composition into separate pieces. Non-limiting examples of kibbles include particulates; pellets; pieces of pet food, dehydrated meat, meat analog, vegetables, and combinations thereof; and pet snacks, such as meat or vegetable jerky, rawhide, and biscuits. A “meat analog” is a meat emulsion product that resembles pieces of natural meat in appearance, texture, and physical structure.

The term “complete and balanced” when referring to a food composition means a food composition that contains all known required nutrients in appropriate amounts and proportions based on recommendations of recognized authorities in the field of animal nutrition, and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO) as of Jan. 1, 2019.

The term “dietary supplement” means a product that is intended to be ingested in addition to a normal diet. Dietary supplements may be in any form, e.g., solid, liquid, gel, tablets, capsules, powder, and the like. In one aspect, they can be provided in convenient dosage forms. In some embodiments, they can be provided in bulk consumer packages such as bulk powders, liquids, gels, or oils. In other embodiments, supplements can be provided in bulk quantities to be included in other food items such as snacks, treats, supplement bars, beverages and the like.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Any embodiment disclosed herein can be combined with any other embodiment disclosed herein unless explicitly and directly stated otherwise.

The present discussion of embodiments, aspects, examples, etc. are independent in that they can apply to all methods and compositions. For example, colostrum used in a food composition can also be used in the method of preventing or treating an allergy of an animal, and vice versa.

### EMBODIMENTS

The present inventors have discovered that a food composition comprising colostrum can provide specific health benefits in an animal. The present methods include enhancing vaccine efficacy for senior animals and treating and/or preventing autoimmune diseases and allergies in animals.

As such, in one embodiment, a method of treating or preventing an allergy, or treating or preventing an autoimmune disease, in an animal, can comprise administering to the animal a composition comprising colostrum.

In another embodiment, a method of enhancing vaccine efficacy in a senior animal can comprise comprising administering to the animal a composition comprising colostrum.

Generally, the colostrum is present in an effective amount to provide the desired health benefit. In one embodiment, the colostrum can be present in the composition in an amount of from about 0.01 weight % to about 1 weight %. In one aspect, the colostrum can be present in the composition in an amount of from about 0.01 weight % to about 0.5 weight %. For supplements, in a different aspect, the colostrum can be present in an amount of from about 0.01 weight % to about 10 weight %.

The present methods can further include identification of specific animals in need of the health benefit. In one embodiment, the method can further comprise identifying the animal having the allergy or identifying the animal is at risk of having the allergy wherein the administration prevents or treats the allergy. In another aspect, the method can further comprise identifying the animal having the autoimmune disease or identifying the animal is at risk of having the autoimmune disease wherein the administration prevents or treats the autoimmune disease. In still another aspect, the method can further comprise identifying the animal in need of a vaccine wherein the administration enhances vaccine efficacy.

Typically, the present compositions can be administered to the animal for a sufficient time to provide the health benefit. In one embodiment, the composition can be administered to the animal on a regular basis. In another embodiment, the composition can be administered long-term. The present compositions are generally food compositions but can also be administered as part of a dietary regime in the form of supplements, treats, sachets, and the like. In one embodiment, the composition is a pet food composition. In another embodiment, the composition is a dietary supplement. In still another embodiment, the composition is a sachet.

Additionally, the compositions disclosed herein can contain other ingredients. In one embodiment, the compositions can contain probiotics and/or prebiotics. Typical probiotics include, but are not limited to, probiotic strains selected from Lactobacilli, Bifidobacteria, or Enterococci, e.g., Lactobacillus reuteri, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain can be selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g., NCC2627), Bifidobacterium sp. NCC2657 or Enterococcus faecium SF68 (NCIMB 10415). Generally, the food compositions can contain probiotics in amounts sufficient to supply from about 10⁴ to about 10¹² cfu/animal/day, in one aspect, from 10⁵ to about 10¹¹ cfu/animal/day, and in one specific aspect, from 10⁷ to 10¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. Pat. No. 6,929,793. In one embodiment, the probiotic can be Enterococcus faecium SF68 (NCIMB 10415). In another embodiment, the probiotics can be encapsulated in a carrier using methods and materials known to skilled artisans.

Prebiotics include any substance that alters microflora composition of the gastrointestinal tract by providing a substrate for growth of microorganisms. Prebiotics include, without limitation, natural and synthesized oligosaccharides, soluble fibers, resistant starch, and gums. The oligosaccharides can be linear or branched. The prebiotic can be specifically chosen for its ability to enhance the survival of the probiotic in the storage container, or in the gastrointestinal tract of an animal. The prebiotic can also be specifically chosen for its ability to enhance the functionality of the probiotic in the animal or to complement the benefits of the probiotic. Generally, prebiotics are administered in amounts sufficient to positively stimulate the healthy microflora in the gut and cause these “good” bacteria to reproduce. Typical amounts are from about one to about 10 grams per serving or from about 5 percent to about 40 percent of the recommended daily dietary fiber for an animal.

As discussed herein, the present composition can include animal digest. Useful animal digests in the present invention can be any animal digest known to skilled artisans included liquid animal digests and dried animal digests. Such digests include any material that results from chemical and/or enzymatic hydrolysis of clean and undecomposed animal tissue. Additionally, dried brewers yeast can be useful in the present invention, which is any dried brewers yeast known to skilled artisans, e.g., the dried, inactive agent that is a byproduct of the brewing industry. Animal digest and dried brewers yeast have been found to enhance the palatability of the compositions, including dietary supplements and food compositions. When present in such compositions, the animal digest comprises from about 1 weight % to about 90 weight % of the composition and the dried brewers yeast comprises from about 1 weight % to about 30 weight % of the composition.

Regarding supplements, the dietary supplements can be prepared as a variety of formulations such as a powder, granule, pellet, or any other appropriate delivery form. In some embodiments, the dietary supplement formulation can be powder containing microencapsulated probiotics within a biopolymer matrix. The powder dietary supplement can be sprinkled over or otherwise applied to and admixed with a food or other composition, particularly a pet food such as dog food or cat food. The powder dietary supplements can be specially formulated for consumption by a particular animal, such as companion animal. In one embodiment, the powder dietary supplement comprises a high concentration of probiotics such that the supplement can be administered to the animal in small amounts, or in the alternative, can be diluted before administration to an animal. A skilled practitioner can devise other routes of administration such as providing the dietary supplement alone or feeding it in, on, or with a pet treat.

The dietary supplements can be formulated for storage for prolonged periods at room temperature or, alternatively, can be refrigerated, freeze dried, or frozen. In one embodiment, the dietary supplement can be stored at room temperature and the probiotics in the dietary supplement remain substantially viable for at least 22 days when stored at room temperature. Predictive modeling showed that such probiotics remain substantially viable despite periodic short-term spikes in storage temperature that can exceed room temperature by up to 60° C.

The compositions of the invention can comprise additional substances such as minerals, vitamins, salts, proteins, amino acids, fibers, condiments, colorants, and preservatives. Non-limiting examples of minerals include calcium, phosphorous, potassium, sodium, iron, chloride, boron, copper, zinc, magnesium, manganese, iodine, selenium and the like, and various salts thereof. Non-limiting examples of vitamins include vitamin A, various B vitamins, e.g., niacin, pantothenic acid, folic acid, biotin, vitamin C, vitamin D, and vitamin K. The compositions may also comprise carotenoids such as alpha-carotene, lycopene, lutein, zeaxanthin and beta-cryptoxanthin. Additional ingredients may also be included, for example, inulin, amino acids, and the like.

In various embodiments, the compositions of the invention may further comprise from about 15% to about 60% crude protein. In one embodiment, the compositions comprise about 40% to about 55% crude protein. The crude protein material may comprise vegetable proteins such as soybean, corn, rice, cottonseed, and peanut, or animal proteins such as casein, albumin, and meat protein. Non-limiting examples of meat protein useful herein include pork, lamb, equine, poultry, fish, and mixtures thereof.

The compositions may further comprise from about 5% to about 40% fat. In one embodiment, the compositions can comprise about 15% to about 30% fat. The compositions may further comprise a source of carbohydrate. The compositions may comprise from about 15% to about 60% carbohydrate. Non-limiting examples of such carbohydrates include grains or cereals such as rice, corn, sorghum, alfalfa, barley, soybeans, canola, oats, wheat, and mixtures thereof. The compositions may also optionally comprise other materials such as dried whey and other dairy by-products.

The compositions may also further comprise at least one fiber source. The compositions may comprise from about 0.5% to about 5% fiber. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, mannanoligofructose, soy fiber, fiber from lupins, arabinogalactan, galactooligosaccharide, arabinoxylan, or mixtures thereof. The fiber source can be a fermentable fiber, as are many of those listed above. Fermentable fiber has previously been described to provide a benefit to the immune system of companion animals. Fermentable fiber or other compositions known to those of skill in the art which provide a prebiotic composition that could enhance the growth of probiotics within the intestine may also be incorporated into the composition to aid in the enhancement of the benefits provided by the present invention to the immune system gastrointestinal system, and general health of an animal.

In some embodiments where the composition is a supplement, to enhance the length of time the supplement can be stored, the dietary supplements can have a total moisture content between about 2% and about 10% by weight of the supplement. In one embodiment, the total moisture content can be less than 5% by weight of the supplement. Similarly, the dietary supplement can have a water activity in the range of 0.20 to 0.6. In one aspect, the water activity can be less than 0.55.

The compositions can be specially formulated for particular animals such as dogs or cats. Similarly, the compositions may be specially formulated for young, adult, or senior animals. In one aspect, the animal can be a senior animal. In another aspect, the animal can be an aging animal. In general, specialized formulations comprise ingredients that meet the energy and nutritional requirements appropriate for particular animals and for particular animals at different stages of development or age, or with specific nutrient requirements related to a disease state.

In one embodiment, the compositions can be formulated for dogs. In another embodiment, the compositions can be formulated for cats.

The skilled artisan can determine the appropriate amount of ingredients to be added to a given formulation. Such factors that may be taken into account include the average consumption of specific types of compositions by different animals, whether the animal that is intended to ingest the dietary supplements has any particular health, wellness, or nutritional requirements, or suffers from a particular disease or disorder, the age, sex, size, or breed of the animal, and the manufacturing conditions under which the composition is prepared. The concentrations of ingredients to be added to the composition can be calculated on the basis of the energy and nutrient requirements of the animal.

As discussed herein, in various embodiments, the present compositions can be pet food compositions. In various embodiments, the pet food composition can be a wet food, a semi-moist food or a dry food. In an embodiment, the pet food composition is one or more components of a blended composition. In some embodiments, the pet food composition is a kibble, and in some embodiments, the pet food composition is a meat analog.

The pet food compositions disclosed herein can be any food formulated for consumption by a canine or feline. In an embodiment, the pet food composition can be a complete and balanced pet food; i.e., provides complete nutrition as defined by the Association of American Feed Control Officials (AAFCO) for a canine or feline.

The pet food compositions disclosed herein can comprise vegetable oil, a flavorant, a colorant and water. Suitable vegetable oils include soybean oil, corn oil, cottonseed oil, sunflower oil, canola oil, peanut oil, safflower oil, and the like. Examples of suitable flavorants include yeast, tallow, rendered animal meals (e.g., poultry, beef, lamb, pork), flavor extracts or blends (e.g., grilled beef), animal digests, and the like. Suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The pet food compositions disclosed herein can optionally include additional ingredients, such as humectants, preservatives, polyols, salts, or combinations thereof. Suitable humectants include salt, sugars, propylene glycol and polyhydric glycols such as glycerin and sorbitol, and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like.

### EXAMPLES

The following non-limiting examples are illustrative of embodiments of the present disclosure.

### Example 1—Canine Study

Thirty senior dogs (7 and 7 plus years old dogs) were randomized into 2 groups based on their age, gender, breed and pre-test fecal IgA level (n=15/group). Dogs in the Control group were fed Super-premium like diet for senior dogs. Dogs in the test group were fed a test diet containing 0.1% Colostrum. After 12-weeks feeding, Lyme vaccine was administered to each dog. After two more weeks (14 weeks after feeding trial started), one boost vaccine was provided to dogs. Multiple blood samples were collected at the beginning of feeding trial, and after vaccinations were given. Antibody titers specific to Lyme vaccine were measured with ELISA kit. Colostrum group had significantly (P<0.05) higher specific antibody titers in serum at multiple time points (week 18, 20, 24 and 28) compared to Control group as shown in Table 1. Moreover, in Colostrum group, the time to reach peak antibody level was significantly (P=0.024) earlier than Control group as shown in Table 2, which means the test group produced peak level of antibody at significantly earlier time compared to Control group.

TABLE 1

| | Weeks after feeding trial | | | | | | | |
|---|---|---|---|---|---|---|---|---|
| | 0 | 16 | 18 | 20 | 24 | 28 | 32 | 36 |
| Colostrum group | 0 | 5.246 | 6.040 | 5.968 | 5.153 | 5.209 | 4.852 | 4.635 |
| (Natural Log of | | | | | | | | |
| vaccine antibody titers) | | | | | | | | |
| Control group | 0 | 4.983 | 5.575 | 5.662 | 4.758 | 4.663 | 4.561 | 4.208 |
| (Natural Log of | | | | | | | | |
| vaccine antibody titers) | | | | | | | | |

TABLE 2

| | Weeks to reach |
|---|---|
| Group | peak concentration |
| Colostrum group | 18.4 |
| Control group | 19.08 |

After 6 months feeding, peripheral blood samples were collected from Labradors in each group (N=5 or N=6). Regulatory T cells (Tregs) were isolated, and suppressive function on effector cells' proliferation (Teffs) by Tregs were evaluated with MTT assay. In Control group, the ratio of (Tregs+Teffs)/Teffs was close to 1, indicating that there is no suppression on Teffs' proliferation by Tregs. However, the ratio of (Tregs+Teffs)/Teffs was 0.806, confirming that Tregs from Colostrum group had significantly (P=0.031) suppressed the proliferation of Teffs as shown in Table 3. Tregs suppress inflammatory events, but they usually poorly function in allergy and autoimmune patients. Colostrum significantly enhances function of Tregs, therefore, colostrum helps to treat or prevent allergy and autoimmune diseases.

TABLE 3

| | Proliferation of |
|---|---|
| Group | Treg + Teff/Teff |
| Colostrum | 0.806252 |
| Control | 0.990572 |

In addition, the secretion of IL-10 by Tregs in Colostrum group was significantly (P=0.021) higher compared to Control group. IL-10 has potent anti-inflammatory and immunosuppressive activities, higher IL-10 levels helps to prevent or treat autoimmune diseases and allergy as shown in Table 4.

TABLE 4

| Group | IL-10 (pg/ml) |
|---|---|
| Colostrum | 226.5596 |
| Control | 82.1824 |

As such, the present methods can include enhancing Treg function in an animal. It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of treating or preventing an allergy, or treating or preventing an autoimmune disease, in an animal, comprising administering to the animal a composition comprising colostrum.

2. The method of claim 1, wherein the colostrum is present in the composition in an amount of from about 0.01 weight % to about 1 weight %.

3. The method of claim 1, wherein the composition is administered to the animal on a regular basis.

4. The method of claim 1, wherein the composition is a pet food composition.

5. The method of claim 5, wherein the pet food composition is formulated for a dog or a cat.

6. The method of claim 1, wherein the animal is a companion animal.

7. The method of claim 1, wherein the animal is a canine.

8. The method of claim 1, further comprising identifying the animal having the allergy or identifying the animal is at risk of having the allergy wherein the administration prevents or treats the allergy.

9. The method of claim 1, further comprising identifying the animal having the autoimmune disease or identifying the animal is at risk of having the autoimmune disease wherein the administration prevents or treats the autoimmune disease.

10. A method of enhancing vaccine efficacy in a senior animal, comprising administering to the animal a composition comprising colostrum.

11. The method of claim 10, wherein the colostrum is present in the composition in an amount of from about 0.01 weight % to about 1 weight %.

12. The method of claim 10, wherein the composition is administered to the animal on a regular basis.

13. The method of claim 10, wherein the composition is a pet food composition.

14. The method of claim 10, wherein the animal is a companion animal.

15. The method of claim 10, wherein the animal is a canine.
